(19) United States
(12) Patent Application Publication
(10) Pub. No.: US 2026/0144717 A1
(43) Pub. Date: May 28, 2026

(54) BIOACTIVE ENDODONTIC CERAMIC SEALER COMPOSITIONS AND METHODS OF USE

(71) Applicant: Inter-Med, Inc., Racine, WI (US)

(72) Inventors: Sara Travia, Racine, WI (US); Alexander D. Johnson, Racine, WI (US); John Baeten, Racine, WI (US); Khongchee Xiong, Racine, WI (US)

(21) Appl. No.: 19/122,178

(22) PCT Filed: Oct. 20, 2023
(86) PCT No.: PCT/US2023/035605

§ 371 Date: Apr. 17, 2025

Related U.S. Application Data
(60) Provisional application No. 63/380,409, filed on Oct. 21, 2022.

(51) Int. Cl. A61K 6/836; A61K 6/20; A61K 6/54; A61K 6/86; A61K 6/876

(52) U.S. Cl. CPC A61K 6/836; A61K 6/20; A61K 6/54; A61K 6/86; A61K 6/876

(57) ABSTRACT

Compositions and methods useful for sealing of root canals are disclosed which provide significant clinical advantages including bioactivity, improved stability and setting and decreased shrinkage. Kits containing the composition and manufacturing processes to create the composition are also disclosed.

### CROSS-REFERENCE TO RELATED APPLICATIONS

This application claims the benefit of U.S. Provisional Application No. 63/380,409, filed Oct. 21, 2022; the entire contents of this application is hereby incorporated by reference herein.

### BACKGROUND OF INVENTION

Human teeth are living structures comprised of both hard and soft tissues. The hard tissue consists of enamel (the hard exterior surface tissue of teeth), dentin (immediately below enamel and helps support enamel), and cementum (a specialized hard tissue that covers the root of a tooth). The dental pulp is generally referred to as soft tissue and primarily resides within the pulp chamber. Pulp tissue consists of many types of cells and connective tissue but also includes blood vessels and nerves that enter the tooth from the apical foramen which is a hole at the apex of a root canal. Generally, a tooth's root canal system refers to the naturally occurring anatomical spaces within the root(s) of a tooth including the pulp chamber.

The root canal system is complex and can become infected in several ways. Commonly this occurs if dental caries are untreated before pulp and root canal infiltration by bacteria. Once a root canal system is infected, many treatment methods can be used depending upon the condition of the patient and approach of the practitioner. Typically, an endodontic procedure called root canal therapy (RCT) is performed to cleanse the root canal system and remove infectious agents.

RCT usually involves three main steps. The first step is called instrumentation and entails opening the tooth and widening the root canal system utilizing a series of semi-rigid metal endodontic files, often times with endodontic irrigants (e.g. sodium hypochlorite) to reduce the debris level created by the endodontic files. This step removes some organic and inorganic material, but its primary goal is to enlarge the root canal(s) to allow for the introduction of small cannula used during the second step, called irrigation. Irrigation entails flushing of the root canal system using small cannula (called irrigation needles) attached to syringes of various aqueous-based endodontic irrigants, including sodium hypochlorite. The goal of the irrigation step is to debride the root canal system and solubilize any remaining debris and smear layer introduced during instrumentation. The third and final step is called obturation, in which the root canal system is filled with various materials. After the endodontic procedure is finalized, a standard dental restorative procedure (i.e. composite filling or crown, among others) is used to complete the structure and aesthetics of the tooth.

RCT of an infected tooth is typically performed in two visits. Between these visits, a disinfectant is placed in the root canal system that destroys remaining microorganisms and prevents reinfection. Calcium hydroxide Ca(OH)₂ is commonly used between treatment appointments due to its antibacterial effect, attributed at least in part to the release of hydroxyl ions over time and their diffusion through dentin. Calcium hydroxide exerts its antibacterial effect in the root canal system as long as a high pH value is maintained. The use of Ca(OH)₂ placed as a disinfectant, however, in the root canal system has certain drawbacks. For example, calcium hydroxide has a low solubility in water and consequently the onset of its disinfecting effect is slow. Moreover, it has to be removed before the canal is filled with a restorative or root-filling material, which is cumbersome since calcium hydroxide particles are hard to retrieve from the complexities of the root canal system. Lastly, a two-visit RCT procedure is not favorable to the dental professional, as the second visit takes up additional non-productive chair time, and for the patient, it is inconvenient.

Although significant advancements have occurred in RCT, there still exists the potential for reinfection of the root canal space which necessitates retreatment or tooth extraction. In an effort to increase the likelihood of one-visit RCT success, and make the obturation step of RCT easier, there has been significant advancement in developing and commercializing “endodontic sealers.” These endodontic sealers are popular. Their use entails first filling the canal(s) with the sealer followed by inserting a master gutta percha cone to a working length that helps physically push the sealer into dentinal tubules and canal complexities. Once in place, the sealers harden over time. Some sealers will harden via a chemical polymerization reaction, for example, from mixing two components together, or via a hydration reaction by extracting water present in the tooth. Currently the most popular sealers are bioceramic sealers that offer some biocompatibility advantages.

Focusing specifically on bioceramic sealers, several patents and commercial products exist. U.S. Pat. Nos. 5,415,547 and 5,769,638 both relate to a material called MTA (Mineral Trioxide Aggregate). MTA is composed of white or grey hydrophilic particles which harden in an aqueous environment, and its major constituents are calcium oxide, aluminum oxide, and iron oxide, silicon dioxide, calcium sulfate, and bismuth oxide (added to improve radiopacity). In an aqueous environment, MTA releases free hydroxide (OH—) through dissociation of calcium hydroxide thereby increasing the local pH. Generally MTA exhibits satisfactory biocompatibility, however, MTA drawbacks include exuding unnatural tooth aesthetics (grey color), difficult ergonomics (multiple components require mixing and have limited working time), long set time within the canal (greater than one hour), and slight expansion once cured in the canal which can put unnecessary stresses on the tooth structure.

U.S. Pat. No. 6,620,232 discloses a ceramic material for dental applications comprising a binder phase consisting of a cement-based system, of which at least 70 vol % consists of calcium aluminate cement in which the grain size is 10 μm or less, characterized in that the material comprises one or more additives adapted to give the material long term dimensional stability properties, said additive comprising Portland cement and/or some other organic silicone-containing phase having a grain size of 0.5-10 μm and/or fine silica having a grain size of less 100 nm, at a content of 1-20 vol % of the material.

U.S. Pat. No. 6,969,424 discloses a method for the production of a chemically bound ceramic material by means of reaction between one or more powdered binding phase and a liquid, a quantity of powder containing said binding phase being suspended in said liquid so that all powder grains are brought into close contact with the liquid, whereupon the slurry thus obtained is drained so that the majority of surplus reacting liquid is removed, and is compacted during final draining before the material hardens by the reaction between said binding phase and the remaining liquid. One or more expansion-compensating additives, adapted to give the material dimensionally stable long-term properties, are mixed into said powder, prior to or in conjunction with its suspension in the liquid.

U.S. Pat. No. 8,475,811 discloses a premixed cement paste for use in medical or dental applications. The premixed cement paste remains fluid when stored in a hermetically sealed condition, but hydrates and hardens to set when placed in a physiological environment. The cement paste includes at least one calcium silicate compound and at least one substantially water-free liquid carrier mixed with the at least one calcium silicate compound: the substantially water-free liquid carrier avoids hydration of the mixture during storage but undergoes exchange with aqueous physiological solutions so that the cement paste hydrates and hardens to set when placed in a physiological environment. This product is commercialized by Brasseler USA (Savannah, GA) as Endosequence BC Sealer (Standard).

U.S. Pat. No. 8,343,271 discloses a hydraulic cement comprising a calcium silicate, at least one phosphate compound, and at least one alkaline-sensitive organic compound. The phosphate compound is included in an amount sufficient to react a major portion of the calcium hydroxide produced during hydration of the cement to hydroxyapatite or other calcium phosphates that co-precipitate with the calcium silicate hydrate to form a composite-like structure on the nano-scale level. The hydroxyapatite content at a reduced pH renders the cement bio-active and suitable for use in medical and dental implants, for example, for replacement bone and tooth material.

U.S. Pat. App. Pub. 2011/0281241 discloses a composition based on calcium aluminate cement (CAC) for application in endodontics, comprising: (a) a cement—Al₂O₃ (>68.5 wt %), CaO (<31 wt %), SiO₂ (0.3-0.8 wt %), MgO (0.4-0.5 wt %), and Fe₂O₃ (<0.3 wt %); (b) additives: dispersant at a content of 0.4 to 0.8% by weight of the cement, a plasticizer at a content of 2.0 to 4.0% by weight of the cement, and a radiopaque agent at a content of 20 to 35% by weight of the cement; and (c) water, wherein a water/cement ratio lies in the range of 0.19-0.24 in the presence of additives. Cementitious product obtained thereof, after setting time, is also disclosed and characterized by enhanced properties when compared to the most used commercial repair cement, MTA.

Irrespective of these developments, there remains a need for improved endodontic sealers that exhibit improved biocompatibility, tissue regeneration, handling, setting, stability, and shrinkage characteristics.

### BRIEF DESCRIPTION OF THE DRAWINGS

FIG. 1 displays volumetric shrinkage data for Formula A, Brasseler EndoSequence BC Sealer Standard, and Brasseler EndoSequence BC Sealer Standard HiFlow. As the results show, Formula A provided the lowest (best) volumetric shrinkage.

FIG. 2 displays set time results for various sealers. Formula A, Formula B, and EndoSeal MTA successfully cured within the test duration (62.7±9.5 min. 37.0±7.4 min and 19.7±1.2 min, respectively) whereas the Brasseler EndoSequence BC Sealers did not set/cure within 300 minutes.

FIG. 3 displays solubility data for Formula A, Brasseler EndoSequence BC Sealer Standard, and Maruchi EndoSeal MTA. As the results show, Formula A provided the lowest (best) solubility value.

### DETAILED DESCRIPTION

The following paragraphs define in more detail the embodiments of the invention described herein. The following embodiments are not meant to limit the invention or narrow the scope thereof, as it will be readily apparent to one of ordinary skill in the art that suitable modifications and adaptations may be made without departing from the scope of the invention, embodiments, or specific aspects described herein.

The present disclosure provides bioactive endodontic ceramic sealer compositions for use in dental treatments, particularly for use in RCT in vivo. In further embodiments, methods of use of the disclosed compositions are described in detail. In yet another aspect, a product by process to create the bioactive endodontic ceramic sealer is disclosed. In an even further aspect, kits containing the composition are disclosed.

In a specific embodiment, the bioactive endodontic ceramic sealer composition is a paste-like, clay-like, or putty-like material that is substantially water free. The composition is generally comprised of at least one bioactive glass that releases biologically active ions in the presence of water, at least one calcium aluminate cement, optionally at least one accelerator that expedites cementation hydration reactions, at least one radiopacifier, at least one strengthening filler, optionally at least one rheology modifier, and a substantially water free liquid carrier. Once the bioactive endodontic ceramic sealer is placed in vivo within the root canal, the set time is less than four hours. In some embodiments, the set time is less than two hours. In some embodiments, the set time is less than one hour.

In certain embodiments, the bioactive glass is a hygroscopic calcium sodium phosphosilicate, for example. Bioglass 45S5 which consists of weight/weight concentration (% w/w) 45% SiO₂. 24.5% CaO, 24.5% Na₂O, and 6.0% P₂O₅. Once in contact with water, the bioactive glass releases biologically active ions such as calcium, phosphate, fluoride, hydroxide, and others deemed clinically advantageous that facilitate remineralization of tooth structures, such as dentin. This remineralization induced by the bioactive glass may, in certain embodiments, offset any sealer shrinkage that commonly plagues conventional sealers. The release of these biologically active ions occurs immediately when in contact with water but can last for days, weeks, months, or longer depending on the bioactive glass composition and particle size. While the bioactive glass is releasing the biologically active ions, the bioactive glass also serves as a structural filler for increasing the strength of the disclosed bioactive endodontic ceramic sealer while also helping to minimize shrinkage. In certain embodiments, the bioactive glass may release antimicrobial chemicals, compounds or agents upon contact with water. In further embodiments, the ions released from the bioactive glass may modify the pH of the surrounding environment to inhibit bacteria, stimulate surrounding cells to promote tissue healing and growth, or reduce inflammation. Besides being biologically active, the ions released from the bioactive glass may also help quicken the cementation hydration reaction and thereby improve the set time of the bioactive endodontic ceramic sealer. Within the disclosed composition, the bioactive glass is preferable at a 325 mesh size (44 um) or smaller. Within the disclosed composition, the bioactive glass may have a weight/weight concentration (% w/w) between 1-20%, and in some embodiments, at about 10%.

The bioactive endodontic ceramic sealer additionally may comprise at least one hygroscopic accelerator. In some embodiments, the hygroscopic accelerator is an alkali metal salt. In some embodiments, the hydroscopic accelerator is lithium chloride. The alkali metal salt may have a weight/weight concentration (% w/w) between 0.0-5%, and in some embodiments between 0.1-1%.

In other embodiments, the bioactive endodontic ceramic sealer may further comprise at least one retarding agent that slows cementation. Retarding agents may slow the cementation process by chelating metal ions that accelerate the curing mechanism, or retarding agents may electrostatically interact with cementation agents, including water, or create a protective layer on the surface of the cement particles to hinder the interaction of water molecules to slow their reaction. Typically, these retarding agents are beneficial for thicker sealers (i.e., more putty-like sealers) to minimize premature setting and ensure satisfactory shelf-life and storage. Suitable examples of retarding agents include metal sulfate salts (such as calcium sulfate), tricalcium phosphate, sodium tripolyphosphate (STPP), borax, borates, boron-containing compounds, lignosulfonates, hydroxycarboxylic acids, organophosphates, metal sulfate salts, among others. In certain embodiments, the retarding agent may release biologically active ions and may also be a strengthening filler. The retarding agent may have a weight/weight concentration (% w/w) between 0.0-10%, and in certain embodiments between 0.5-7.5%.

The calcium aluminate cement may have a weight/weight concentration (% w/w) between 10-70%, and or between 20-30% within the bioactive endodontic ceramic sealer. The main phase of the calcium aluminate cement may be calcium aluminate (CA; CaAl₂O₄), and calcium dealuminate (CA₂; CaAl₄O₇), while the secondary phase of the calcium aluminate cement may be C12A7 and alpha-A. In certain embodiments the alumina content within the calcium aluminate is at a weight/weight concentration (% w/w) between 50-80%, between 60-75%, or about 70%. In certain embodiments the calcium oxide content within the calcium aluminate is at a weight/weight concentration (% w/w) between 15-50%, between 20-40%, or about 30%. Preferably, all other oxides (e.g. SiO₂, Fe₂O₃, TiO₂, MgO, Na₂O, K₂O, SO₃) are at a weight/weight concentration (% w/w) less than 10.0%, and in some embodiments, all other oxides are at a weight/weight concentration (% w/w) less than 1.0%. These various characteristics may result in a hardened endodontic cement sealer that exhibits favorable porosity that better mimics dentin and results in improved integration with anatomical tissue. Once in contact with water inside the root canal in vivo, the described calcium aluminate cement directly forms the calcium aluminate hydrate C3AH6 [3CaO·Al₂O₃·6 H₂O], which is unique to the disclosed composition as two other hydrates (CAH10) [CaO·Al₂O₃·10 H₂O] or C2AH8 [2 CaO·Al₂O₃·8 H₂O]) are usually formed when using calcium aluminate cements. This is advantageous as C3AH6 is highly stable and the least soluble of all calcium aluminate hydrates. Furthermore, the bioactive endodontic ceramic sealer provides enhanced compressive strengths once set compared to existing sealers on the market. In certain embodiments, the resulting compressive strength is >15 MPa, >25 MPa, or >50 MPa. Within the disclosed composition, the calcium aluminate cement may have a 325 mesh size (44 μm) or smaller. In certain embodiments, at least one transitional metal silicate may also be added to the composition as additional cementation ingredients.

The disclosed bioactive endodontic ceramic sealer is also comprised of at least one radiopacifier at a weight/weight concentration (% w/w) between 20-60%, and in some embodiments between 40-50%. In some embodiments, the radiopacifier is a transition metal oxide, for example, zirconium oxide or bismuth oxide, or a compound containing iodine, barium, tantalum, gold, ytterbium, or tungsten. Preference is given to compounds and materials that are colorless, white, or are slightly colored. In certain embodiments of the disclosure, the radiopacifier is added to the bioactive endodontic ceramic sealer preferably at a concentration that provides a radiopacity value of at least 7 mmAl, at least 8 mmAl, or at least 9 mmAl.

In some embodiments, the bioactive endodontic ceramic sealer contains a rheology modifier that may also optionally serve as a strengthening filler to improve the set sealer's compressive strength and minimize shrinkage during cementation/curing. In some embodiments, the rheology modifier is silicon dioxides (for example, fumed silica). In some embodiments, the rheology modifier has a weight/weight concentration (% w/w) between 0.5-5%. In some embodiments the rheology modifier has a weight/weight concentration between 1-3%. In some instances, the rheology modifier may introduce thixotropic properties to the bioactive endodontic ceramic sealer. Additional thickeners may be added depending on the consistency of the sealer formulation desired for clinical use. The disclosed bioactive endodontic ceramic sealer may have a viscosity of 5-2000 Pa·s, of 50-800 Pa·s, or of 200-500 Pa·s. In certain embodiments, a much thicker bioactive endodontic ceramic sealer resembles a paste or putty which preferably has a viscosity of 1,000-200,000 Pa·s. 1,500-75,000 Pa·s, and or in some embodiments 2,000-15,000 Pa·s. The bioactive endodontic ceramic sealer has a viscosity allowing it to be easily introduced into the root canal for improved penetration into dentinal tubules and canal complexities for improved mechanical bonding to the dentin tooth structure. In certain aspects, the bioactive endodontic ceramic sealer may be non-Newtonian or pseudoplastic. Suitable thickeners can include silicates, glasses and polymers, such as polystyrene, polypropylene, polyethylene, polyacrylates, polyacrylamides, polyvinyl alcohol, copolymers and surfactant combinations, and barium glass.

The bioactive endodontic ceramic sealer is substantially water free and instead obtains its fluid/liquid characteristics from a non-aqueous carrier to which all the solid ingredients are added and kept in suspension. In some embodiments, this carrier contains at least one alcohol (hydroxyl) group, and may be a diol or ether compound, and in certain instances the carrier may also be hygroscopic to help promote water absorption once placed within the body. In certain embodiments, the carrier has a weight/weight concentration (% w/w) between 10-60%, or between 35-50%. Suitable carriers include propylene glycol, polyethylene glycol, glycerin, ethanol, among others. In certain embodiments, the carrier is propylene glycol. In further embodiments, the carrier is polyethylene glycol (PEG). Suitable PEG molecular weights include PEG molecules between 200 g/mol and 10,000 g/mol. In further embodiments, a mixture of propylene glycol and PEG is preferred. Suitable propylene glycol to PEG mixtures may include ratios between 10:1 and 1:10.

In some embodiments, the bioactive endodontic ceramic sealer may additionally comprise surfactants that decrease the sealers' surface tension thereby facilitating easier introduction into the root canal and improve penetration into dentinal tubules. Preferably, the surfactants are non-ionic such that they do not interact with the cementation reaction or biologically active ions released from the Bioglass. Examples of suitable surfactants include nonionic surfactants, such as alcohol ethoxylates, polyoxyethylene glycol octylphenol ethers, polyoxyethylene glycol alkylphenol ethers, polyoxyethylene glycol sorbitan alkyl esters, sorbitan alkyl esters, copolymers of polyethylene glycol and/or propylene glycol, Poloxamers, sodium sterates, fluorosurfactants, among others. Optionally, the bioactive endodontic ceramic sealer includes at least one humectant. Optionally, the bioactive endodontic ceramic sealer includes at least one lubricant.

Generally, all ingredients should exhibit long-term biocompatibility for safety and toxicity considerations. Furthermore, the selection of ingredients within the bioactive endodontic ceramic sealer may also exhibit more favorable biocompatibility than other sealers commercially available. In certain embodiments, all ingredients within the bioactive endodontic ceramic sealer composition are at a 325 mesh size (44 um) or smaller. This size restriction helps the disclosed sealer composition by 1) expediting the cementation reaction through increased surface area of reactants, and 2) ensuring composition particles are small enough for extrusion through small cannulas for delivery within the root canal and subsequent penetration into dentinal tubules and canal complexities. In some embodiments, the pH of water in contact with the disclosed bioactive endodontic ceramic sealer is alkaline (i.e. pH is >7). In some embodiments, the pH is >9. In some embodiments, the pH is >10. Alkalinity from strong bases (for example, from sodium hydroxide), however, is not an ideal source of hydroxide ions as it is known in the art that this may lead to “cement cancer” and a lack of cementation/hardening. Therefore, in certain embodiments weak bases may be utilized within the composition.

The ratio between composition ingredients may contribute to the observed favorable properties of the bioactive endodontic ceramic sealer. In some embodiments, the ratio between carrier, cement, and additives is between 1:1.25:2 and 1:2.5:6. In certain embodiments, the ratio between carrier, cement, and additives is about 1:1.5:2.5.

In further aspects of the disclosure, the bioactive endodontic ceramic sealer composition provides a suitable shelf life of between 6 and 48 months when stored at room temperature without displaying characteristics of chemical instability or significant decreased clinical efficacy. The bioactive endodontic ceramic sealer may optionally be stored in a hermetically sealed pouch to help minimize moisture absorption and help extend shelf life. An example of a hermetically sealed pouch may be a foil pouch comprising layers of polyethylene terephthalate (PET), low density polyethylene (LDPE), aluminum foil, linear low density polyethylene (LLDPE), among others. In some embodiments, the hermetically sealed pouch is between 0.002 inches and 0.008 inches thick. In some embodiments, the hermetically sealed pouch has a water vapor transmission rate between 0.01 mg/100 in²/24 hours and 0.8 mg/100 in²/24 hours (ASTM F1249), and preferably has an oxygen transmission rate between 10 nL/m²/24 hours and 10 uL/m²/24 hours. An example of chemical instability would be the formation of a precipitate, or significant phase separation, during storage or the premature hardening/cementation of the material within the syringe or packaging that renders the composition less effective or useless (for example, if the composition hardens in the syringe and cannot be extruded for clinical use).

In certain aspects, the bioactive endodontic ceramic sealer may be packaged in various syringes or containers sized between 0.1 mL-100 mL, and in some embodiments syringes sized between 0.5 mL-5 mL. Syringe packaging can be advantageous as application tips can be mated directly to the syringes of the bioactive endodontic ceramic sealer for immediate application to the root canal. In some embodiments, it may be beneficial to have the bioactive endodontic ceramic sealer composition stored in separate containers (e.g. two separate bottles, dual barrel syringe, etc.) and mixed by the user immediately prior to use.

The packaging of the components must be compatible for long-term storage (months to years). Satisfactory plastic resins for the packaging material may include, but are not limited to, polypropylene, polycarbonate, polyethylene, styrene acrylonitrile, methyl methacrylate-acrylonitrile-butadiene-styrene, poly-cyclohexylenedimethylene terephthalate glycol, among others.

In certain aspects, the bioactive endodontic ceramic sealer is provided as an item within a kit. In some embodiments, the kit may comprise any one or more of the following components: application tips, application brushes, mixing tips, mixing vessels, empty syringes, an instructions for use, mixing wells or other single use vessels, a dental etchant or etchants, a dental adhesive or adhesives, a dental primer or primers, a dental composite or composites, a dental cement or cements, an endodontic irrigant or irrigants, among other common dental and endodontic products.

The method of using the bioactive endodontic ceramic sealer typically comprises introducing the bioactive endodontic ceramic sealer into the root canal system through the use of various application tips attached to a syringe containing the sealer therein. Usually after the root canal is filled with the bioactive endodontic ceramic sealer material, a master gutta percha cone is inserted to working length which helps push and introduce the bioactive endodontic ceramic sealer material into dentinal tubules and canal complexities (e.g. isthmuses, lateral canals, etc.) while also ensuring the sealer reaching the entire apical third of the root canal(s). Once introduced in the root canal, the bioactive endodontic ceramic sealer starts to set and harden by reacting with water present naturally within the tooth.

The method of manufacturing the bioactive endodontic ceramic sealer is as follows: first placing the substantially water free carrier in a mixing vessel then adding and mixing the solid powdered components into the substantially water free carrier. The solid powdered components may be added all at once to the substantially water free carrier followed by mixing to homogenize the mixture. Alternatively, the solid powdered components may be added sequentially with mixing steps between each component to homogenize the mixture. Or another alternative is that the solid powdered components are individually added in increments with mixing between each increment, and this process is completed for each solid powdered component. In certain embodiments, the bioactive endodontic ceramic sealer may be manufactured using 1-30 mixing steps, and in some embodiments, using 2-20 mixing steps. Adding and mixing of the substantially water free carrier and solid powdered component in these different processing techniques impacts the end performance of the sealer which may provide benefits for various clinical uses. In other words, the same bioactive endodontic sealer composition can be mixed in these different processes to yield materials displaying different properties such as viscosity/film thickness, tackiness, and set time. In certain embodiments, it is advantageous for manufacturing of the bioactive endodontic ceramic sealer to be performed in a bladeless dual asymmetric centrifuge mixer. In some embodiments during mixing of the composition, the composition does not exceed 70° C. In some embodiments, the composition does not exceed 60° C. during mixing.

### Definitions

For purposes of interpreting this specification, the following abbreviations, terms and definitions will apply and whenever appropriate, terms used in the singular will also include the plural and vice versa. In the event that any definition set forth below conflicts with any document incorporated herein by reference, the definition set forth below shall control.

The term “room temperature” or ambient temperature as used herein refers to common ambient temperatures ranging from about 18° C. to about 27° C.

The term “treating” refers to administering a therapy in an amount, manner, or mode effective to improve a condition, symptom, or parameter associated with a disorder. In some aspects, treating refers to the treatment of a dental ailment such as an infected tooth.

The term “substantially” as used herein means to a great or significant extent, but not completely.

As used herein, “a”, “an”, “the”, “at least one”, and “one or more” are used interchangeably.

The terms “comprises” and variations thereof do not have a limiting meaning where these terms appear in the description and claims.

The term “patient” or “subject” refers to mammals and humans. Thus, in one aspect, the subject is a mammal, or a mammal in need thereof. In one aspect, the subject is a human, or human in need thereof. In one aspect, the human or human in need thereof is a medical patient. The subject can be from ˜0 years of age to 99 years of age or older.

The term “in vivo” generally means in a living subject.

The term “composition” generally refers to the chemical makeup of certain embodiments of the disclosed invention and is synonymous with “formula”.

The term “chemical stability” generally refers to a composition or formula that remains in chemical equilibrium for a period of time without significant reactivity. In some instances, this stability can be observed visually, for example, if there is not a change in the composition's state, such as observing the formation of a visible solid precipitate over time or phase separation. Said differently, the observation of a visible precipitate or phase separation within a composition or formula over time would indicate the initial formula was chemically instable. The precipitate then formed due to the composition desiring to became more chemically and thermodynamically stable.

The term “endodontic” generally refers to inside the tooth. The term “endodontic procedure” is generally synonymous with “root canal therapy”, or “root canal procedure”, and refers to a treatment of an infected tooth to cleanse the root canal system and remove the infection.

The term “cementation” generally refers to the process of a material containing cement to react with water to set or harden (i.e. via a hydration reaction). Specifically in the context of this disclosure, the bioactive endodontic ceramic sealer contains a calcium aluminate cement which reacts with water to transition from a fluid state to a rigid state via this hydration reaction. In the context of this disclosure, “cementation” is generally synonymous with “setting”, or “hardening” and these types of words are used interchangeably throughout the disclosure.

The term “smear layer” is known to those of skill in the art of dentistry and refers to the complex accumulation of soluble and insoluble organic and inorganic debris resulting from the mechanical preparation of a tooth surface. The smear layer includes cutting debris, tooth particles, microorganisms, necrotic material, and other substances resulting from preparation, and can include a superficial layer on the surface of a prepared tooth along with a layer or layers that are packed into the adjacent dentinal tubules at varying depths.

The term “sealer” generally refers to materials that are used as part of the obturation step of an endodontic procedure to help seal the canal. This term is generally synonymous with “endodontic sealer” and includes bioceramic sealers and non-bioceramic sealers. In certain applications, this sealer can be thin as a readily free flowing liquid, whereas in other applications, this sealer may resemble a putty, paste, or clay-like material.

The term “root canal system” generally refers to the naturally occurring anatomical spaces within the root(s) of a tooth including the pulp chamber.

The term “bioactive” generally refers to a material exhibiting properties that actively interact with surrounding biological tissue and environment. This term contrasts with “biologically inert” which describes a material that does not interact with the surrounding biological tissue and environment.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### EXAMPLES

Table 1 summarizes the chemical constituents that comprise the disclosed bioactive endodontic ceramic sealer. Additionally, Table 2 provides example formulas or compositions of the disclosure.

TABLE 1
Chemical constituents that comprise the disclosed bioactive endodontic ceramic sealer

| | | | Embodiment 1 | Embodiment 2 |
|---|---|---|---|---|
| | | | Concentration | Concentration |
| Ingredient | Example | Function | (w/w %) | (w/w %) |
| Calcium sodium phosphosilicate | Bioactive glass | Bioactive | 5-20% | ~10% |
| Alkali metal salt | LiCl | Accelerator | 0.0-5% | 0.1%-1% |
| Metal sulfate salt | Calcium sulfate | Retarding agent | 0.0-10% | 0.5-7.5% |
| Calcium aluminate cement | CAC | Cement | 10-70% | 20-30% |
| Diol | Propylene glycol | Carrier | 10-60% | 35-50% |
| Transitional metal oxide | Zirconium oxide | Radiopacifier | 20-60% | 40-50% |
| Silicon dioxides | Fumed silica | Rheology | 0.5-5% | 1-3% |
| | | modifier or | | |
| | | strengthening | | |
| | | filler | | |

TABLE 2
Example formulas or compositions that comprise the
disclosed bioactive endodontic ceramic sealer

| | | Formula A | Formula B |
|---|---|---|---|
| Ingredient | Classification | (w/w %) | (w/w %) |
| Bioglass 45S5 | Calcium sodium | 9.50% | 9.50% |
| | phosphosilicate | | |
| Lithium chloride | Alkali metal salt | 0.15% | 0.00% |
| Calcium aluminate cement | Cement | 27.00% | 24.50% |
| Propylene glycol | Carrier | 20.00% | 11.35% |
| Zirconium oxide | Radiopacifier | 41.85% | 41.85% |
| Fumed silica (Aerosil 200) | Rheology modifier | 1.50% | 2.80% |
| Calcium sulfate | Retarding agent | 0.00% | 5.00% |
| Tricalcium phosphate | Retarding agent & | 0.00% | 5.00% |
| | biologically active | | |
| | material | | |

| | | |
|---|---|---|
| TOTAL | 100.00% | 100.00% |

The composition of the bioactive endodontic ceramic sealer results in many clinical advantages for the patient and dental professional compared to existing products on the market including, but not limited to: 1) decreased shrinkage post-curing/hardening. 2) optimized viscosity and handling for improved penetration into dentinal tubules and canal complexities. 3) improved set time/cure time, 4) improved wettability and sealability to the root canal surface, 5) decreased water solubility, 6) antimicrobial properties. 7) satisfactory radiopacity. 8) improved biocompatibility, and 9) improved stability and shelf-life.

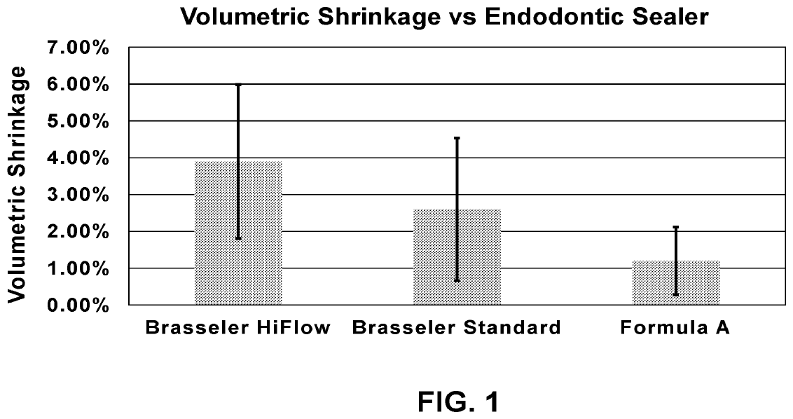

To evaluate the shrinkage characteristics of the bioactive endodontic ceramic sealer, Formula A was subjected to a shrinkage study following the methodology described in ISO 6876:2012 “Dentistry-Root canal sealing materials” (hereafter referred to as ISO 6876). Briefly, a humidity chamber was constructed that maintains ≥95% relative humidity at 37° C. Gypsum molds made from type 2 dental plaster were manufactured as identified in ISO 6876 and were conditioned within the humidity chamber enclosure for at least 24 hours. The conditioned molds were then separately filled with Formula A and comparison products currently on the market. Brasseler EndoSequence BC Sealer Standard and Brasseler EndoSequence BC Sealer HiFlow (Brasseler USA, Savannah, GA). A metal straight edge razorblade was used to ensure the sealer material was completely flush with the top of the gypsum mold. The filled molds were placed in the enclosure for 24 hours to ensure total curing of the sealer materials. Molds were sectioned in half using a wet-diamond saw, such that a cross section of the cured sealer material and gypsum mold were visible. A calibrated micrometer, with measurement accuracy to 0.001 mm, was then used to measure the height of the gypsum mold and the height of the cured sealer sample at the middle of the cured specimen. Sealer volumetric shrinkage was then determined by calculating the difference between the two measurements and the known height of the cured sealer specimen. A sample size of three was used in this study. Results are shown in FIG. 1 and demonstrate that Formula A had the lowest shrinkage percent (1.2%±0.9%) compared to Brasseler EndoSequence BC Sealer Standard (2.6%±2.0%) and Brasseler EndoSequence BC Sealer HiFlow (3.9%±2.1%). The lower shrinkage percent exhibited by the disclosed bioactive endodontic ceramic sealer. Formula A, is clinically advantageous as less shrinkage-related stress will be applied within the tooth and root canal system following curing of the sealer material. This feature helps mitigate potential tooth fracturing, reinfection, etc. In certain embodiments, the bioactive endodontic ceramic sealer results in volumetric shrinkage values <5.0%, <2.0%, or <1.5%. In certain embodiments, the volumetric shrinkage is analogous referred to autogenous shrinkage.

The bioactive endodontic ceramic sealer also exhibits improved flowability and handling characteristics that are helpful for improved penetration into dentinal tubules and canal complexities. Testing followed the methodology detailed in ISO 6876. Briefly. 0.05±0.005 mL of sealer was placed on one glass plate. A second glass plate was placed on top of the sealer sample after 180 seconds. A 100 gram mass was placed on top of the second glass plate. After 10 minutes, the mass was removed and the maximum and minimum diameters of the compressed sealer disc were measured using a calibrated caliper. Formula A was evaluated in this test and Maruchi's EndoSeal MTA (Maruchi USA, Yorba Linda, CA) was tested for comparison. A sample size of three was used in this study. Table 3 shows the results of this study. Compared to EndoSeal MTA, Formula A resulted in a 10% increase in disc diameter (21.3 mm versus 23.4 mm, respectively) which supports that Formula A has improved flowability and handling characteristics. In certain embodiments, the bioactive endodontic ceramic sealer results in a disc diameter >22 mm when tested following ISO 6876 and in some embodiments results in a disc diameter between 22-25 mm.

TABLE 3
Flow test results of endodontic sealers following ISO 6876. Compared
to EndoSeal MTA, Formula A resulted in a 10% increase in disc diameter
(21.3 mm versus 23.4 mm, respectively) which supports that Formula
A has improved flowability and handling characteristics.

| Material | Average Disc Diameter (mm) |
|---|---|
| Formula A | 23.4 |
| Maruchi EndoSeal MTA | 21.3 |

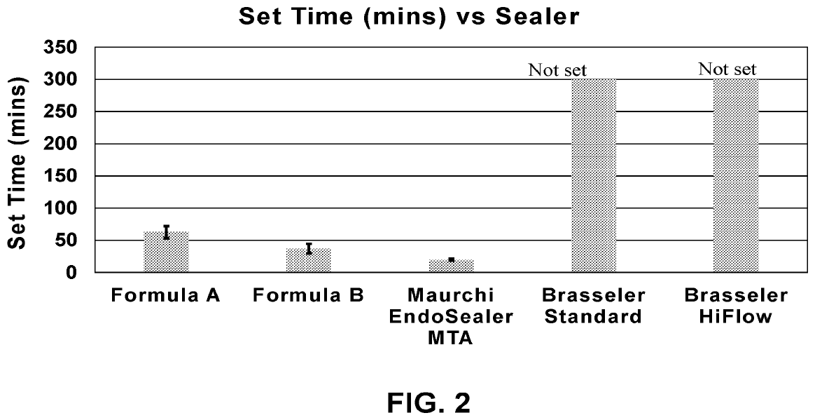

To evaluate the set time characteristics of the bioactive endodontic ceramic sealer, Formula A was subjected to a shrinkage study as described in ISO 6876 along with Brasseler EndoSequence BC Sealer Standard & Maruchi EndoSeal MTA products. Briefly, a humidity chamber was constructed that maintains ≥95% relative humidity at 37 C. Gypsum molds made from type 2 dental plaster were manufactured as identified in ISO 6876 and were conditioned within the humidity chamber enclosure for at least 24 hours. The conditioned molds were then separately filled with the various sealer specimens. At determined time intervals, samples were removed from the chamber enclosure. A Gilmore indenter (100.0±0.5 g. with a flat end diameter of 2.0±0.1 mm) was then placed on the sealer specimen to determine if the sealer was cured or not. If an indentation was visible on the sealer surface, then the sealer sample was placed back into the humidity chamber for additional time to reach a cured state. This process was continued until an indentation was no longer visible which indicates the sealer sample is sufficiently cured. Testing was performed in triplicate and testing was stopped at 300 minutes. FIG. 2 summaries the results of this test. Formula A and EndoSeal MTA successfully cured within the test duration (62.7±9.5 min and 19.7±1.2 min, respectively) whereas the Brasseler EndoSequence BC Sealers did not cure within 300 minutes. Although the EndoSeal MTA cured faster than Formula A, this might not always be advantageous as certain endodontic procedures can take longer than ˜20 minutes and in certain situations dental professionals may need to re-access the root canal system within that time. In other words, having a sealer cure too fast might be clinically disadvantageous, therefore, the cure time or set time of the disclosed bioactive endodontic ceramic sealer has been optimized for clinical use. In certain embodiments, the bioactive endodontic ceramic sealer cures (hardens) between 10-600 minutes. In some embodiments, the sealer cures between 15-120 minutes. In some embodiments, the sealer cures between 30)-90 minutes.

Another set time study was conducted where the Bioglass 45S5 in Formula A was replaced with the same amount of 0.7 μm barium glass which is a biologically inert material strengthening filler (this substituted formula is hereafter identified as Formula A′). Following the same test methodology as described above. Formula A′ resulted in an average set time of 71.3 minutes which is 14% longer than the set time of Formula A. These results illustrate how the bioactive glass positively impacts and helps accelerate the cementation/hardening reaction of the calcium aluminate cement once in the presence of water.

Film thickness of the bioactive endodontic ceramic sealer (Formula A) compared to EndoSeal MTA was evaluated following methods detailed in ISO 6876. Briefly, the combined thickness of two glass plates was first measured with a calibrated caliper. The plates were separated, and the sealer sample was placed onto the center of one glass place. The other glass plate was then placed on top of the sealer. After 180 seconds, a load of 150±3 N was applied vertically on the top plate such that the sealer completely fills the area between the two glass plates. After 10 minutes, the combined thickness of the two glass plates plus sealer was measured with the calibrated caliper to calculate the sealer's film thickness. Testing was performed in triplicate. Results are shown in Table 4. Compared to EndoSeal MTA, Formula A resulted in a 25% decrease in film thickness (26 um versus 35 um, respectively) which supports that Formula A has improved flowability characteristics that better wet the canal surface and create a better seal with the root canal system. In certain embodiments, the bioactive endodontic ceramic sealer has a film thickness <30 um when tested following ISO 6876. In some embodiments, the sealer has a film thickness of about 25 um. In applications where a thicker, more putty-like sealer is advantageous, the bioactive endodontic ceramic sealer may have a film thickness between 50 um-250 um, and in some embodiments between 75 um-125 um.

TABLE 4
Film thickness results of endodontic sealers following ISO 6876.
Compared to EndoSeal MTA, Formula A resulted in a 25% decrease
in film thickness (26 um versus 35 um, respectively) which supports
that Formula A has improved flowability characteristics that
better wet the canal surface and create a better seal with the
root canal system. For more putty-like sealer compositions, Formula
B was compared to Endosequence BC Putty & Endosequence BC Putty
Fast Set (Brasseler USA, Savannah, GA). Here, Formula B provided
a lower film thickness value of 101 um compared to the other
products which supports that Formula B has improved flowability
characteristics that better wet the canal surface and create
a better seal with the root canal system.

| Material | Consistency | Film Thickness (um) |
|---|---|---|
| Formula A | Flowable sealer | 26 |
| Maruchi EndoSeal MTA | Flowable sealer | 35 |
| Formula B | Putty-like sealer | 101 |
| BC Putty | Putty-like sealer | 126 |
| BC Putty Fast Set | Putty-like sealer | 169 |

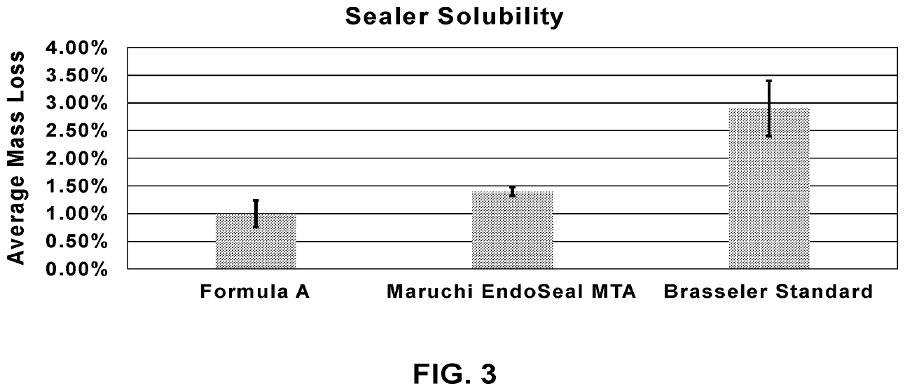

Solubility and disintegration of the bioactive endodontic ceramic sealer (Formula A) compared to EndoSeal MTA was evaluated following methods detailed in ISO 6876. Data for Brasseler's EndoSequence BC Sealer Standard was obtained from published peer-reviewed literature (J Endod. 2013 October; 39(10):1281-6) which also followed ISO 6876 test methodology. Briefly, sealer specimens were cured in a split ring mold as detailed within ISO 6876 and initial mass measurements were obtained on a calibrated analytical scale. Once cured, samples were placed in a covered petri dish with 50 mL deionized water for 24 hours at 37° C. Next, the petri dish was emptied through a fluted filter to catch the cured sealer samples, which were then washed three times with 5 mL deionized water. The sealer samples were then placed in an oven at 110° C. to evaporate water to constant mass. Final mass measurements were then recorded. Solubility percentages (w/w %) were then calculated by comparing the initial and final masses of the sealer samples. Results are summarized in FIG. 3. As the data shows, Formula A resulted in the lowest mass loss/solubility percentage (1.0%±0.2%) compared to EndoSeal MTA (1.4%±0.1%) and EndoSequence BC Sealer Standard (2.9%±0.5%). Because endodontic sealers are intended for long-term use within teeth (years), having a low solubility is important and advantageous to ensure mechanical integrity, stability, and longevity of the sealer and endodontically-treated tooth. In certain embodiments, the bioactive endodontic ceramic exhibits <5.0% (w/w %) solubility in water. <2.0% (w/w %) solubility in water, or exhibits <1.0% (w/w %) solubility in water.

To investigate the impact of manufacturing and mixing on the bioactive endodontic ceramic sealer, Formula B was manufactured following two different processes. In the first process, the substantially water free carrier (propylene glycol) was added to a mixing vessel followed by adding all solid powdered components (all other ingredients in Formula B) to the substantially water free carrier. Four one-minute mixes at 2300 RPM (Speedmixer, FlackTek, Landrum SC) were used to homogenize the mixture. In the second process, the substantially water free carrier (propylene glycol) was added to a mixing vessel. Each solid powdered component was added in three increments, with a one-minute mix at 2300 RPM used between each increment addition. The order of addition for the solid powdered component is not critical besides calcium aluminate cement being added as the last component. Since there are six solid powdered components used in Formula B, this resulted in 18 total mixes of one minute at 2300 RPM. Two additional one-minute mixes at 2300 RPM were used to ensure homogenization of the mixture at the end. The set time of the resulting Formula B mixtures was evaluated following ISO 6876 methods previously described. Results are summarized in Table 5 and show that incrementally adding the solid components, coupled with further mixing, resulted in a material that exhibited considerably faster set time.

TABLE 5
Set time results of Formula B following ISO 6876 for
different manufacturing processing techniques.

| Formula | Processing Technique | Total Mixes | Set Time (min) |
|---|---|---|---|
| Formula B | Solids added to | 4 mixes, 1 min, | 83 |
| | carrier and mixed | 2300 RPM | |
| Formula B | Each solid added | 20 mixes, 1 | 37 |
| | incrementally to | min, 2300 RPM | |
| | carrier with mixes in | | |
| | between increments | | |

What is claimed is:

1. A bioactive ceramic composition for use as an endodontic sealer, comprising:
at least one bioactive glass;
at least one calcium aluminate cement;
at least one radiopacifier;
at least one strengthening filler; and,
at least one substantially water-free carrier.

2. The composition of claim 1, wherein the at least one bioactive glass comprises a hygroscopic calcium sodium phosphosilicate.

3. The composition of claim 1, wherein the at least one bioactive glass comprises Bioglass 45S5 at a concentration between 5%-20% wt/wt %.

4. The composition of claim 1, wherein the at least one bioactive glass comprises a calcium sodium phosphosilicate serving as a structural filler that releases biologically active ions.

5. The composition of claim 1, further comprising an accelerator, wherein the accelerator is lithium chloride.

6. The composition of claim 1, wherein the calcium aluminate cement is at a concentration between 20-30%.

7. The composition of claim 1, wherein the calcium aluminate cement comprises about 70% alumina and about 30% calcium oxide.

8. The composition of claim 1, wherein the strengthening filler is also a rheology modifier.

9. The composition of claim 1, wherein the strengthening filler improves the sealer's compressive strength and minimizes composition shrinkage during curing.

10. The composition of claim 1, wherein the strengthening filler is at a concentration between 1-3%.

11. The composition of claim 1, further comprising at least one of the following: a surfactant, a humectant, or a lubricant.

12. The composition of claim 1, wherein all ingredients are at a 325 mesh size or smaller.

13. The composition of claim 1, wherein the composition cures (hardens) between 30-90 minutes in the presence of water.

14. The composition of claim 1, wherein the composition results in a volumetric shrinkage of less than 1.5% once cured.

15. The composition of claim 1, wherein the at least one bioactive glass accelerates setting of the calcium aluminate cement.

16. The composition of claim 1, wherein the composition exhibits <1.5% solubility in water.

17. The composition of claim 1, wherein the composition exhibits antimicrobial properties in the presence of water.

18. The composition of claim 1, wherein the composition retains its stability for more than 60 days without phase separation or the formation of a precipitate.

19. A method of sealing the root canal of a tooth, comprising:
introducing a composition of any one of claim 1 into a root canal or root canals.

20. An endodontic kit, comprising:
a composition of claim 1; and,
application tips.
